(19) United States
(12) Patent Application Publication
(10) Pub. No.: US 2004/0248750 A1
(43) Pub. Date: Dec. 9, 2004

(54) Soap bar wrapper

(76) Inventors: Alan Nimmey, Cenon (FR); Todd VanGordon, Basking Ridge, NJ (US); Steve Johnson, Basking Ridge, NJ (US); Randy Kliment, Milford, NJ (US)

Correspondence Address: Colgate-Palmolive, Patent Department, 909 River Road, P O Box 1343, Piscataway, NJ, 08855-1343, US

(21) Appl. No.: 10/479,197

(22) PCT Filed: May 30, 2002
(86) PCT No.: PCT/US02/16914

Related U.S. Application Data
(62) Division of application No. 60/295,380, filed on Jun. 1, 2001.
(62) Division of application No. 60/313,119, filed on Aug. 17, 2001.
(62) Division of application No. 60/343,379, filed on Dec. 21, 2001.

(51) Int. Cl. A61K 7/50

(52) U.S. Cl. 510/141

(57) ABSTRACT

Soap bars that are not of essentially a square or rectangular shape, but of a generally rectangular shape, can be packaged using a single plastic film wrapper. This plastic film preferably is biaxially oriented polypropylene of from about 60 micron to about 180 microns. It can be a monolayer or a multilayer laminated. However, any plastic film having a Taber stiffness of more than about 5 and preferable about 7 to 20 can be used. In order to assist in making folds to form the end walls, the film material can be weakened by being thinned, perforated or slit at the fold points or lines. In addition, in order to make the longitudinal sealin the package bottom surface the seal plate can be of a shape that is complimentary to the shape of the bottom surface of the soap bar.

### FIELD OF THE INVENTION

This invention relates to wrappers for shaped soap bars. More particularly this invention relates to a single plastic wrapper for a shaped generally rectangular soap bar.

### BACKGROUND OF THE INVENTION

Soap bars are produced in various shapes. These range from round, to square, to rectangular to elliptical and variations on these shapes. Some soap bar shapes are easy to wrap while others present a measure of difficulty. Square, rectangular and circular shaped bars are relatively easy to package. They can be wrapped in a single relatively thin plastic film folded at the ends and heat sealed. These also can be packaged in a flow wrap. In this type of wrapper the ends are fin sealed rather than being folded and sealed. However elliptical shaped bars and generally rectangular shapes having curved edges and curved primary surfaces present packaging difficulties in other than flow wrap packaging. Examples of generally rectangular soap bars are shown in US Des. 345,817; US Des. 346,241; US Des. 348,539 and US Des. 348,541. These design patents are incorporated herein by reference. These have a rectangular-like shape having main top, bottom, side and end surfaces that are curved. Further the transition of one surface to another surface is curved. The soap bars are rectangular-like but with non-planar top, bottom and front and rear connecting surfaces. The end surfaces may have a flat area for sealing but otherwise usually are curved. This includes ovoid shaped soap bars since they also have an elongated type of structure. The transition from one surface to another surface preferably also is curved. This curving gives the soap bar a pleasing, aesthetic shape. Such soap bars are described herein as generally rectangular shaped soap bars.

In order to wrap such bars so that they have a substantially rectangular shape, a stiffener reinforcing element is conventionally used to shape the package. The stiffener reinforcing element is a piece of relatively stiff material such as paperboard, paperboard coated with plastic or plastic that is wrapped laterally around the soap bar with the ends of the soap bar being open. Then the soap bar and the stiffener reinforcing element are surrounded by a wrapper which is folded at its ends and the flaps of the folds heat sealed, one to the other. There also is a longitudinal seal of the film on the bottom surface of the package.

A problem in the use of a stiffener reinforcing element is that it requires the handling and use of two films. The stiffener reinforcing element film and the wrapper film. The packaging process and cost can be reduced with the use of only one film, the wrapper film. That is, the stiffener reinforcing element films is deleted. It has been found that this can be done if a particular wrapper film is used. This film will be sufficiently stiff to provide a rectangular shape to the packaged soap bar, but yet be able to be readily folded and sealed to produce the package.

### BRIEF DESCRIPTION OF THE INVENTION

It has been found that a reinforcing element stiffener is not required if a plastic wrapper of about 60 micron to about 180 micron is used, and preferably about 90 micron to about 150 micron. This plastic can be of a monolayer or laminate multi-layer construction. Such a plastic has a sufficient thickness to form and maintain a generally rectangular shape, but yet a thickness where the film can be folded to form the end side panels without the need for optional fold enhancement techniques. Preferably it will be a thermoplastic film to aid in the sealing of the film. In such a wrapping of soap bars there also is a longitudinal lap seal on the lower surface of the package with the end seals having folds and the folds sealed. There usually are heat seals since thermoplastic films are used. An adhesive can be used to form such heat seals in conjunctions with the thermoplastic films.

The films of this invention have a Taber stiffness of more than about 5 to form the package and are sufficiently flexible so that end panels can be formed and sealed. Preferably the Taber stiffness is about 8 to 20. A preferred film is biaxially oriented polypropylene. This can be a monolayer or a multi-layer of two or more layers. However any other sealable plastic films having similar Taber stiffness can be used. These usually will have a thickness in the 60 micron to 180 micron range. However, it is the stiffness that primarily will control use. The films should have a Taber stiffness of about 7 to 20 which is equivalent to that of about 60 micron to about 180 micron biaxially oriented polypropylene film.

In one optional embodiment the films are weakened by thinning, perforating, or slitting at the point that a fold is to be made in the film to make the end panels for the soap bar package. This will aid in making folds at the proper fold lines and will also provide a technique for making more consistent and crisp folds.

In a further optional embodiment to aid in making the longitudinal bottom seal a seal plate is used that is complementary to the shape of the bottom surface of the soap bar. In a preferred embodiment the bottom surface of the soap bar is concave and the seal plate surface is convex. The concave curvature being such that the convex surface of the seal plate contacts the concave curvature of the soap bar surface. In this way a good longitudinal heat seal can be made.

### BRIEF DESCRIPTION OF THE DRAWINGS

FIG. 1 is a top plan view of a sheet of film having fold assisting perforations.

FIG. 2 is a top plan view of a sheet of film having fold assisting slits.

FIG. 3 is a side elevational view of a seal plate having a seal surface complimentary to the bottom surface of a soap bar.

FIG. 4 is a side elevational view if a soap bar on the sealing plate of FIG. 3.

FIG. 5 is a side elevational view of a wrapped soap bar being sealed on the seal plate of FIG. 3.

FIG. 6 is a view of a soap bar package with folded end seals.

### DETAILED DESCRIPTION OF THE INVENTION

The present invention although useful with many objects will be described with regard to a soap package that does not require a stiffener to wrap generally rectangular soap bars and will be set out in more detail with reference to the preferred embodiments. A generally rectangular soap bar is one described above that has an elongated rectangular-like shape inclusive of an ovoid shape, with main surfaces that are curved and preferably curved in the transition from one main surface to another. Examples are shown in the above U.S. Design Patents which have been incorporated herein by reference. It is an aesthetically pleasing shaped soap bar and one that is relatively easy to grip and to use. However, it is difficult to package in other than a carton, flow wrap, or a two component package that requires both a stiffener sheet laterally around the soap bar and a wrapper film that fully encloses the soap bar.

It has been found that a one piece package can be used to package soap bars and other objects that are not generally rectangular in shape. A single plastic film having a thickness of 60 micron to about 180 micron and preferably about 90 micron to about 150 micron, and a Taber stiffness of more than about 5, and preferably about 7 to about 20 can be used. This is an average Taber Stiffness for both directions. This film is sufficiently stiff to form a rectangular-like package for the soap bar or other object. This film can be opaque, transparent or translucent. The film also can be printed with a decoration or information about the product.

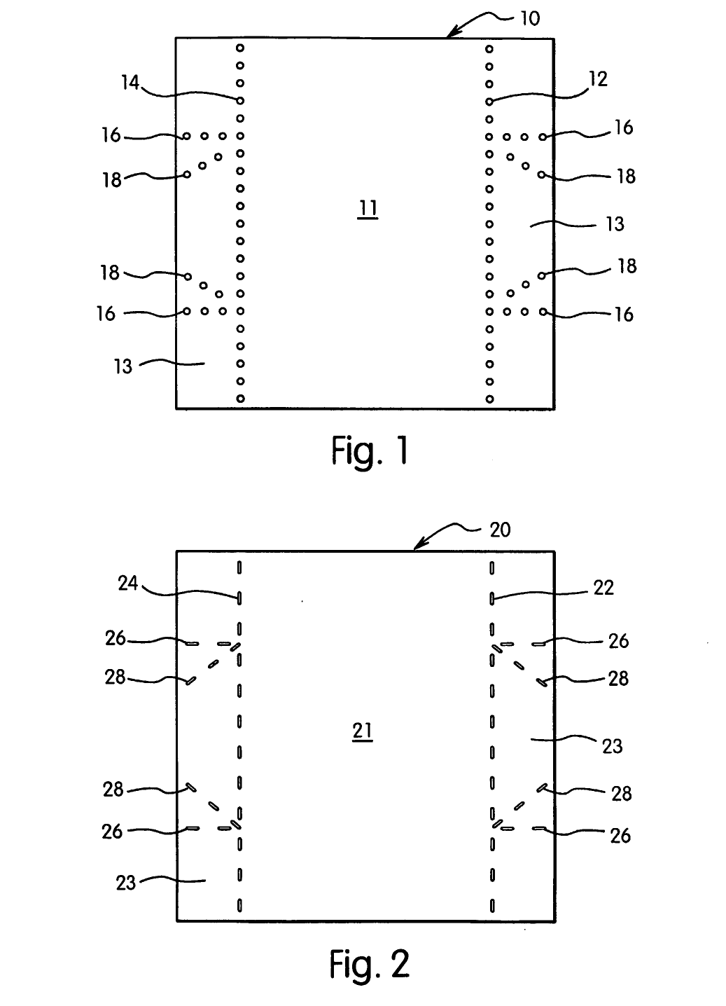

FIG. 1 shows a soap bar that is packaged in a 104 micron biaxially oriented polypropylene film. The soap bar is generally rectangular, having curved surfaces where the top and bottom surfaces intersect the longitudinal side surfaces and the lateral end surfaces. The present film will provide a package with essentially flat top and bottom surfaces. At the corners of there may be some chamfering of the film. However, this chamfering is in itself decorative.

It also is a part of the present invention to form weakened points or lines for the end folds of the soap wrapper to be formed. In wrapping the soap bar a piece of film is cut and is wrapped laterally around a soap bar. Where the longitudinal ends of the film overlaps to provide the seal on the bottom wall surface. The ends are then folded inward to form end seals. This folding inward consists of first folding inward the ends of the sidewalls and then folding inward over the folded ends of the sidewalls the ends of the top wall and bottom wall. This folding of the ends of the top wall and the bottom wall can be in either order. However, it is preferred to first fold the end of the bottom wall and then the end of the top wall. After the folds have been made the overlapping pieces are sealed, one to the other to form the side. This usually will be through a heat sealing through the use of a thermoplastic plastic material which may have a coating to assist in bonding.

When the film has a relatively high Taber stiffness and consequently is relatively thick it is advantageous to weaken the film where folds are to be made. FIG. 1 shows a plurality of perforations 12 and 14 in film 10 where there is to be a fold to form the end walls 13. The portion 11 will form the top surface, bottom surface and the connecting sidewall surfaces. Perforations 16 and 18 are fold lines in the end walls to form the overlapping seals as described above. FIG. 2 shows the use of slits to form the weakened areas. The film 20 has slits 22 and 24 which are fold points of the area 21 from the end wall areas 23. These perforations or slits can be made mechanically or electronically with the use of lasers. The area 21 will form the top surface, bottom surface and the connecting sidewalls. The area 23 will form the folded and sealed end walls. Slits 26 and 28 are weak points/lines to also assist in making the folds to form the end walls. This weakening assists the automatic packaging equipment to make the folds at the right position. A further embodiment is that in place of perforations and slits, the film can be weakened by being thinned at these fold points or lines to about half its thickness. This technique will preserve the barrier function of the package's main body panels.

In addition to making seals in the end walls, there is the need to make a longitudinal seal in the bottom of the package. In this seal the film is overlapped and the overlapped portions heat sealed together. This is conventionally accomplished by the wrapped soap bar passing over a heated seal plate. This seal plate usually is a flat plate. However, for soap bars with a shaped lower surface it is preferred that the seal plate have a surface that is complimentary to the shape of the bar lower surface.

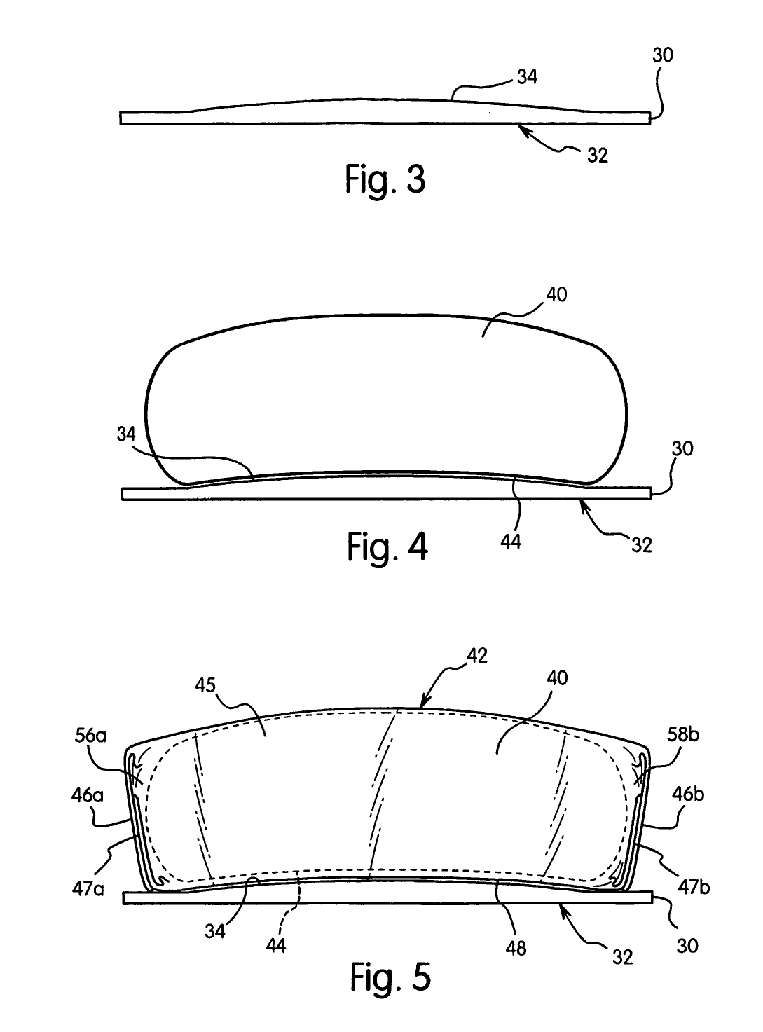

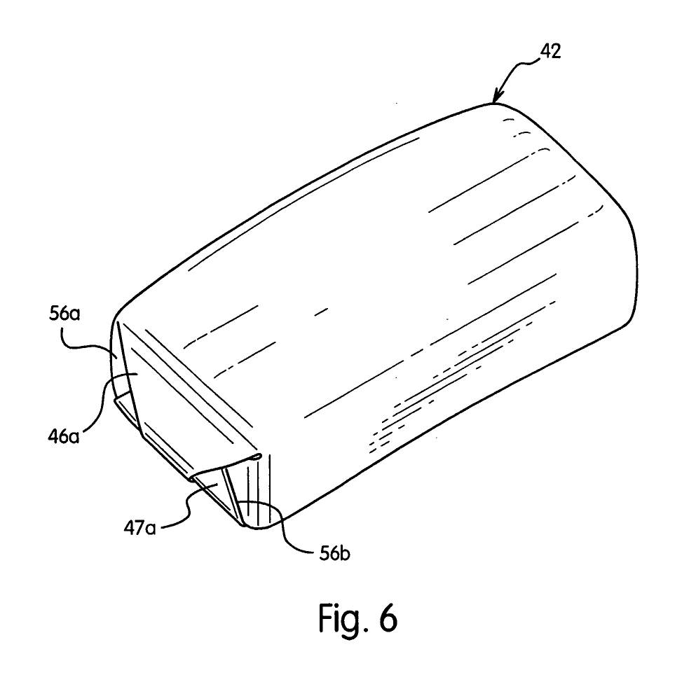

FIG. 3 shows a seal plate 30 having a planar lower surface 32 and a convex upper surface 34. As is seen in FIG. 4 this convex surface 34 is complementary to the concave lower surface 44 of soap bar 40. FIG. 5 shows a packaged soap bar 40 on seal plate 30 with the lower surface 48 of film 42 being sealed. The wrapped soap bar moves over the seal plate surface 34 at a rate of about 60 to 600 soap bars a minute. Front side 45 of the soap wrapper 42 is shown in this view. Also shown are the folds 46(a) and 47(a) on the left side end of the soap bar package and 46(b) and 47(b) or the right side end of the soap bar package. Further shown are the parts 56(a) and 58(a) of the front side 45 that are first folded in with the similar parts from the rear side surface to form part of the end seal. This shows rear side end 56(b) that is folded in to form the left side. The end seals can be made at about the time that the bottom surface seal is being made, or can be made prior to or subsequent to making the bottom surface seal. As described above the film material to form the end walls is folded. The end film material from the front and rear sidewalls is folded inwardly first, and then the top surface and the bottom surface film materials are folded in an overlapping arrangement and sealed. This end seal configuration is further shown in FIG. 6.

The film material can be essentially any thermoplastic having a thickness of about 60 microns to about 180 microns and preferably about 90 microns to about 150 microns. The Taber stiffness should be more than about 5, and preferably about 7 to 20. The useful plastics are polyethylenes, polypropylenes, vinyl polymers such as polyvinyl chloride and polyvinyl acetate, ethylene-propylene copolymers, acrylic polymers, styrenes and polyesters such as polyethylene terephthalate. Functional equivalents of these materials also can be used. A preferred material is biaxially oriented polypropylene. These thermoplastics can be transparent, translucent or opaque and can contain fillers and blowing agents.

We claim:

1. A wrapper soap bar comprising a generally rectangular soap bar wrapped laterally with a thermoplastic film having a thickness of about 60 micron to about 180 micron and a Taber Stiffness of more than about 5, the longitudinal ends of said soap bar enclosed by a folded over edge portion of said film.

2. A wrapped soap bar as in claim 1 which said thickness is about 90 micron to a about 150 micron and a Taber Stiffness of about 7 to 20.

3. A wrapped soap bar as in claim 1 wherein said thermoplastic film is polyethylene.

4. A wrapped soap bar as in claim wherein said thermoplastic film polypropylene.

5. A wrapper soap as in claim 4 wherein said thermoplastic film is biaxially oriented polypropylene.

6. A wrapped soap bar as in claim 1 wherein said thermoplastic film is polyvinyl chloride.

7. A wrapped soap bar as in claim 1 wherein said thermoplastic film is polyethylene terephthalate.

8. A wrapped soap bar as in claim 1 wherein said folded over end portions of said film are weakened at some of the fold lines.

9. A wrapped soap bar as in claim 8 wherein said folded over end portions of said film are weakened at each fold line.

10. A wrapped soap bar as in claim 8 wherein said fold lines are weakened by perforations.

11. A wrapped soap bar as in claim 8 wherein said fold lines are weakened by slits.

12. A wrapped soap bar as in claim 8 wherein said fold lines are weakened by thinning said film at said fold line.

13. A method of wrapping a generally rectangular soap bar having a shared-bottom surface comprising wrapping a film laterally around said soap bar and overlapping the edges of said film that extend longitudinally across a bottom surface of said soap bar, placing said soap bar on a sealing plate that has a shape that is complementary to the shape of the bottom surface of said soap bar, folding the film at a longitudinal end of said soap bar, and sealing said overlapping edges of film and the folded film at the longitudinal end.

14. A method as in claim 13 wherein the bottom surface of said soap bar is concave in shape and said sealing plate is convex shape.

15. A method as in claim 14 wherein said film is folded at both longitudinal end of said soap box.

16. A method as in claim 14 wherein the overlapping edges of film and the folded film at the longitudinal end are sealed at about the same time.

17. A method as in claim 14 wherein the overlapping edges of film and the folded film a t a longitudinal end are sealed sequentially.

18. A method as in claim 14 wherein said film is a thermoplastic film having a thickness of about 60 micron to about 180 micron and a Taber stiffness of more than about 5.

19. A method as in claim 18 wherein said film is selected from the group consisting of polyethylenes, polypropylenes, vinyl polymers, acrylic polymers, styrenes and polyesters.

20. A method as in claim 19 wherein the film is biaxially oriented polypropylene.
